(19) United States
(12) Patent Application Publication
(10) Pub. No.: US 2021/0044440 A1
(43) Pub. Date: Feb. 11, 2021

(54) Blockchain-based clinical trial management

(71) Applicant: Jebril Filali, Oakland, CA (US)

(72) Inventor: Jebril Filali, Oakland, CA (US)

(21) Appl. No.: 16/938,967

(22) Filed: Jul. 25, 2020

Related U.S. Application Data
(60) Provisional application No. 62/883,079, filed on Aug. 5, 2019.

(51) Int. Cl. H04L 9/32

(52) U.S. Cl. CPC H04L 9/3247; H04L 2209/56; H04L 2209/38; H04L 9/321

(57) ABSTRACT

Disclosed are methods and systems for securely managing clinical activity. A system and method may include forming a value representing the clinical activity including a digital signature, distributing the value to a blockchain having contracts and distributing the value to a third party.

### CROSS-REFERENCE TO RELATED APPLICATIONS

The present application claims the benefit of priority under 35 U.S.C. § 119 from U.S. Provisional Patent Application Ser. No. 62/883,079 entitled “Blockchain-based clinical trial management” filed on Aug. 5, 2019, the disclosure of which is hereby incorporated by reference in its entirety for all the purposes.

### BACKGROUND

Clinical trials continue to increase in complexity and scope. The complexity stems from the need to comply with extensive, and consistently evolving regulations; the increasing costs to conduct clinical trials; and the effort and nuances of governing clinical trials.

Current technology has contributed to the challenge by promoting isolated databases from different sources. Data is scattered across different applications and legacy systems don't communicate with each other. Often, data. is transferred from one system to another manually, thus incurring a high probability of errors in the process. Because clinical trials are highly regulated, the integrity of the data and the safety of the patient is essential for its success. Indeed, lack of reproducibility, related to a wide range of scientific misconduct, from errors to frauds, compromises the outcome of a clinical trial.

### BRIEF DESCRIPTION OF THE DRAWINGS

A detailed description will be made with reference to the accompanying drawings:

FIG. 1 illustrates an example of an architecture for managing a clinical trial with one or more contracts using a blockchain in a more secure way.

FIG. 2A, FIG. 2B, and FIG. 2C together are a flowchart illustrating an example of capturing clinical trial activity into a datastore by validating the clinical trial activity with one or more contracts using a blockchain

FIG. 3A, FIG. 3B, and FIG. 3C together are a flowchart illustrating an example of verifying the integrity of clinical trial data from a datastore and a blockchain.

### DETAILED DESCRIPTION

The detailed description set forth below might be practiced without these specific details and is intended to provide a thorough understanding of the subject matter. Structure and components may be represented in block diagram or flowchart for the purpose of clarity and to avoid obscuring the concepts of the subject matter. However, the subject technology may he practiced without these specific details.

Blockchain can be used to validate and store a chain of blocks where successive blocks are linked to earlier blocks via a hash link.

Blockchain may seem an ideal solution to manipulate and store sensitive private health information. However there are major drawbacks to using a public or private blockchain to validate or store private health information. Public blockchain may expose private health information to none authorized parties, may fail to meet regulatory and compliance requirements and expose users to legal liability. Further, private blockchain don't offer enough security. An attacker may take control of the full system and conspire to corrupt the data.

The disclosed methods and systems address the problem by providing a blockchain having contracts for manipulating and validating private health information along with an third party service for distributing the data and optionally one or more digital signatures.

Further, disclosed are methods and systems to securely manage a clinical trial using ways that guarantee the integrity of the data, and adherence to a set of rules defined by a protocol.

In a general aspect, methods and systems to manage the clinical trial may include one or more of the following features:

In one aspect, clinical trials generate an activity that is captured in a datastore along with the proof that demonstrates the validity of processes and data.

The clinical trial generates an activity that may be managed by one or multiple processes that contain one or multiple clinical trial steps. A process designates a series of activities related to the clinical trial. The activities are conducted to achieve a clinical goal regulated by a strict set of rules and requirements as defined by the protocol. A clinical trial step may designate a particular step within a process.

Processes and clinical trial steps may be managed with software designed to help sponsors to conduct the clinical trial including: An electronic data capture. A clinical trial management system. Or any other tool used to manage the clinical trial. The datastore may be a repository for persistently storing and managing collections of data.

The datastore may include a transaction. A transaction may symbolize a unit of work performed within a datastore management system. A transaction may provide an “all-or-nothing” mechanism which means that each work performed in a datastore may either be complete or have no effect. The datastore may include database systems like MySql, Postgres.

In another aspect, by validating an activity through contracts on top of a private blockchain.

Blockchain technology manages transactions into an ongoing chain forming a record that cannot be changed. In simple terms, a blockchain may he a distributed ledger with immutable storage. What that means is that the ledger may be append-only storage: new information is added but the previous information cannot be modified, removed or updated. All the contents in the blockchain may be arranged in blocks where each block may be added with a link to the previous using a cryptographic algorithm that guarantees the integrity of the chain. No one is able to add a falsified block without making the whole chain invalid.

A private blockchain may maintain an access control layer to allow only certain authorized participants to perform specific functions.

A sponsor may vet participants of the private blockchain in order to participate in a consortium of nodes. A node may be an electronic device that records the transactions occurred in a blockchain. A consortium of nodes may be a collection of nodes with the same copy of the data. The nodes in the blockchain may communicate via a secured network and may be restricted to authorized users through an access control layer. The participants may be trusted parties authorized to receive sensitive data from the clinical trial for validation and management by operating one or multiple nodes of the private blockchain.

The participants can include: The sponsor himself can run one or multiple nodes of the private blockchain. By doing so the sponsor may not need to rely on the service provider to guarantee the integrity of the data and the processes. An accredited healthcare institution can run one or multiple nodes of the private blockchain. A hospital or other healthcare provider can run one or multiple nodes of the private blockchain. Any other trusted third party vetted by the sponsor can run one or multiple nodes of the private blockchain.

A contract may be a computer code running on top of a blockchain that facilitates, verifies, and enforces a set of rules between multiple parties who agree to interact with each other. Practically, the contract has a list of steps. A step can involve multiple parties and define a number of conditions that can be used to define any complex interaction between the parties. When the step is fully validated the contract is automatically executed and goes to the next step. The contract can formalize the relationships between people and institutions and the assets they own based on conditions—rights and obligations—to which each party has consented. The contract may be used to control and validate crucial clinical trial activity directly on top of the blockchain and define new models of collaboration between the parties involved. Blockchain records every step of a clinical trial. Contracts manage processes to ensure clinical trial events are happening in chronological order—limiting data falsification and data beautification.

The clinical trial step resulting from the validation of a contract can be associated with a proof stored in the blockchain. The proof for a clinical trial step may establish key factual elements including

Data integrity: The information content of a clinical trial step corresponds exactly to its intended purpose in the recorded process of the clinical trial without any corruption or data tampering.

Actor Non-Repudiation: The actor responsible for the clinical trial step may be recorded in such a way that the source of the information cannot be repudiated by the actor.

Proof of anteriority: The time at which the clinical step occurred may be provable.

Proof of context: Where the clinical trial step belongs relative to the context of all other steps in the clinical trial processes.

Proof of process: The rules and code used to validate each step of the process may be provable.

An example of private blockchain that can be used with this method may be hyperledger.

An authorized administrator may manage the contract using a programming language and an electronic device able to communicate with the blockchain. For example, in hyperledger, the authorized administrator can leverage a contract specification called chaincode to program a set of contracts to control, manage and verify the clinical trial steps.

The method does not constrain the administrator with a specific contract. It's up to the specification designed by the clinical trial team to decide the extent of the contract.

Example of Steps Controlled by One or More Contracts Include:

Protocol: Describes how a clinical trial may be conducted—the objective, design, methodology. Every version of the protocol may he stored in the blockchain allowing the contract to drive the accuracy of the procedures.

Consents: All clinical trials require a patients consent. The clinical trial consent provides a summary of the trial, including its purpose, the procedures, schedule, potential benefits and risk. The contract prevents the deliverance of procedures and treatment if the patient did not agree to enroll, or if the patient was not informed of a new protocol version.

Statistical analysis plan: This plan may assure that analyses to evaluate all planned study hypotheses are conducted in a scientifically valid manner and that all decisions are documented including methods for handling missing data and multiplicity of data, definition of harm events, and sample size to ensure that the clinical trial has enough power.

Collaboration/Disintermediation: May provide a medium to make the patient's data more secure and fine grain controlled. This technique may use differential privacy and can allow very modem usage of the data while complying to regulations. Researchers can access anonymized data from electronic health records without compromising the patient's confidentiality or compliance to regulations. The blockchain may define where is the data and what the managers can access. It may connect to a tiers partner (hospitals, healthcare providers) and may record a log in the audit trail.

Internet of Things (IoT): IoT devices connected via contracts may allow for continuous transfer and collection of data based on prespecified terms in blockchain while still ensuring regulatory compliance.

Supply chain: May manage and trace clinical supply distribution, usage, and return via contracts.

Contracts are programmed in such a way that failure to abide by the rules enforced by the code revokes the request and notify the participants of the source of infringement.

In another aspect, by using hash function algorithms to verify whether some input data map onto a given hash value.

A hash function is any function that maps an input data of arbitrary size onto a hash. It's a one-way function, infeasible to invert. Hash function may be used to create a representation of digital data without the need to disclose the content of the data. Thus, the representation can be stored in a public database controlled by an independent third-party and used later as a proof of the integrity of the data.

In another aspect, by using digital signatures to identify the authenticity of data. A digital signature is a cryptographic algorithm used for verifying the authenticity of digital data. A digital signature can give a recipient good reason to believe the message was originally created by the author (non-repudiation) and was not altered (integrity).

Digital signatures may be used to assert the authenticity and integrity of data related to clinical trials. Digital signatures verify the authenticity and permissions of a user involved in a clinical trial.

Digital signature schemes may be composed of multiple algorithms including: A key generation algorithm that may randomly produce a private key and a corresponding public key. A signing algorithm that may produce a signature when given a message and a private key. A signature verifying algorithm that may either accept or reject the message's claim to authenticity given the message, public key, and signature.

Given the message's claim, the public key, and the signature, an algorithm verifies the authenticity of a signature. Some examples of signing schemes may include: RSA (Rivest-Shamir-Adleman)

In another aspect, participant identities may be managed with a public key infrastructure. Managing participant identities may include a set of hardware, software, policies, processes and procedures to create, manage, distribute, use, store, and revoke digital certificates and public-keys—also known as a public key infrastructure

A digital certificate, also known as a public key certificate or identity certificate, is an electronic document that might be used to prove the ownership of a public key. The digital certificate may include information about the key, information about the identity of its owner and might contain the digital signature of the entity that has verified the certificate's contents. The certificate's subject may communicate securely with a valid signature. Public key infrastructure helps establish the identity of people, devices, and service.

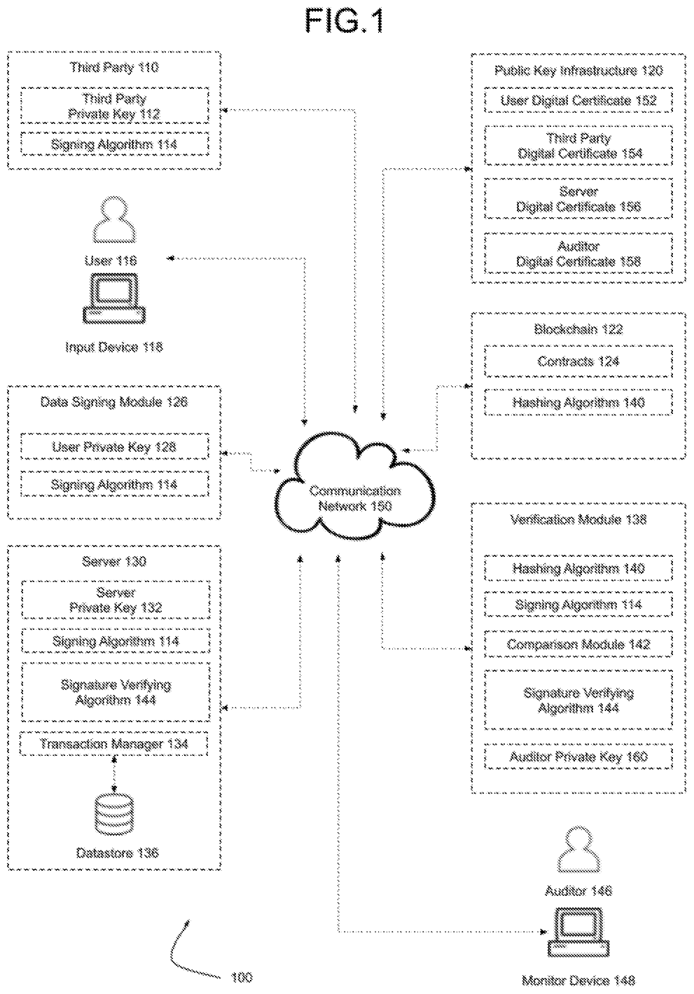

FIG. 1 illustrates an example of an architecture for managing a clinical trial in a more secure way with one or more contracts using a blockchain.

Architecture 100 includes third party 110, user 116 with input device 118, data signing module 126, server 130, public key infrastructure 120, blockchain 122 and verification module 138.

Third party 110, user 116 with input device 118, data signing module 126, public key infrastructure 120, blockchain 122 and verification module 138 are in communication with each other over a communication network 150 (eg. private network, public network, VPN, internet)

User 116 may use input device 118 to manage data related to the clinical trial.

A user 116 may be a person that can perform a clinical trial activity. He may own an identity represented by a protected user private key 128, and a user digital certificate 152 that might have been distributed by a public key infrastructure. The status of the clinical trial may be displayed on input device 118 via a graphical interface that presents the advancement of the clinical trial and a set of optional or required actions for the user 116. Actions may be simple as read or may result in updating the data related to the clinical trial. As explained above these set of actions are regrouped in processes and clinical trial steps. These actions can vary for each user depending on its role, permissions, and phase of the clinical trial.

Some examples of actions include: A user captures data related to a patient's vital status or reports an adverse event at a healthcare facility. A user updates a status related to a query originating from a clinical trial manager. A user reports the shipping of investigational medicine to a hospital.

Data signing module 126 may include user private key 128 and signing algorithm 114. User 116 may use input device 118 to communicate with data signing module 126 via communication network 150.

Data signing module 126 may be used by user 116 with input device 118 to generate a unique signature associated with some clinical trial activity by using signing algorithm 114 and user private key 128. The signing algorithm 114 generates a signature that can be used to prove the identity of a request and therefore improve the security of a system. Input device 118 can be a personal computer, a smartphone or any other electronic device able to interact with the system. Data signing module 126 can be located in different areas including directly colocated within input device 118 or in a remote server that serves the interface via HTML.

Third party 110 may include third party private key 112 and signing algorithm 114. Third party 110 may provide a process for securely keeping track of the creation and modification times of data related to a clinical trial activity.

Third party 110 may receive only the signature generated by the data signing module 126 to improve the privacy of data. To generate a time stamped signature, third party 110 may provide the signature generated by data signing module 126, third party private key 112 and the current time to signing algorithm 114. The resulting signature can be used to prove the time of the clinical trial activity generated by user 116 and originally signed by data signing module 126. Third party 110 may be delegated to a trusted third-party or a public blockchain (e.g. Bitcoin, Ethereum). Because Third party 110 may receive only a cryptographic hash of the data, therefore more security and safety with using a public blockchain or third-party service to generate a proof of time thus improving the privacy of the date while guaranteeing the proof-of-time.

Blockchain 122 may include contracts 124. Blockchain 122 may be a private-distributed ledger that supports contracts 124. Contracts 124 may validate the clinical trial activity with the set of rules defined by the clinical trial protocol. Clinical trial activity can be arranged in one or more processes and each process can be made of one or more clinical trial steps. Each clinical trial step has one or more validation constraints that can be verified by contracts 124.Furthennore, clinical trial steps can be arranged in one or more graph processes where a node may represent a clinical trial step and an edge may represent a chronological dependency between two clinical trial steps. The graph can describe the dependencies of clinical trial steps related to each other forming a coherent structure describing the chronology of the audit trail.

Contracts 124 validate clinical trial steps and store a proof of validity by generating a graph of hash representing the interdependencies of proof between one or multiple clinical trial steps.

The contract 124 generates a proof for the clinical trial step by establishing contextual and cumulative proof using a hash chain. A proof for a clinical trial step can be produced by generating a digest, or hash link, resulting from passing all the data related to the clinical trial step along with the signatures, timestamp and by including one or more of its previous step along with the other key factual elements to hashing algorithm 140. By optionally including one or more previous step proofs, we form a chain of cumulative proofs to ensure the contextual proof. A clear and provable step sequence may be established by cryptographically linking the hash link of previous clinical trial steps in the following clinical trial step of the process. Sharing a cumulative proof of a clinical trial step includes sharing the proof of all its previous proofs. Cumulative proofs are tamper-proof. To change the result of one would invalidate the whole process. Hash links may be stored in a persistence storage in each node of blockchain 122 and can he used to prove the validity of a clinical trial.

By capturing the proof of the previous clinical trial steps in each new clinical trial step, the order of events and their interdependencies are captured in such a way that a sponsor can prove that each clinical trial step has been executed in accordance to the requirements determined by previous actions

Server 130 includes server private key 132, signing algorithm 114, transaction manager 134, signature verifying algorithm 144, and datastore 136. Server 130 may be an electronic system that has a datastore supporting transaction management.

As explained above, a transaction may symbolize a unit of work performed within a datastore management system. A transaction may provide an “all-or-nothing” mechanism which means that each work performed in a datastore may either be complete or have no effect. The electronic system may be a machine or device that performs calculations and operations based on instructions provided by a software or hardware program. Examples : server, personal computer, laptop computer. Datastore 136 may be used to store data related to the clinical trial. Transaction manager 134 may be used to open, commit or rollback a transaction on datastore 136. Server 130 may verify incoming requests by verifying the signature of requests with the signature verifying algorithm 144. Server 130 may commit a transaction in datastore 136 if the data related to a clinical trial activity have been validated by contracts 124.

Auditor 146 may use monitor device 148 to verify the validity of the clinical trial.

An auditor 146 may be a person that may have the permissions to read and verify the data. Auditor 146 may be a representative of a regulatory authority. Auditor 146 may be a consultant that audits the quality of the clinical trial. Monitor device 148 can be a personal computer, a smartphone or any other electronic device able to interact with the system. Monitor device 148 can be used to fetch data from datastore 136 and blockchain 122.

Verification module 138 may include hashing algorithm 140, signing algorithm 114, comparison module 142, signature verifying algorithm 144, and auditor private key 160.

Verification module 138 may be used by auditor 146 to verify die validity of the clinical trial. Signature verifying module 144 can be used to verify the signature generated by the other components of the system Hashing algorithm 140 can be used to reconstruct the graph of hash links constructed by contracts 124 and comparison module 142 can be used to compare them.

The verification module may be used to verify a clinical trial and verify the proof of process and clinical trial step related to a clinical trial including : The chronological order and time of every step. The integrity of the process and data. The proof of the users involved with every step . The proof of context and proof on process.

Verification module 138 can operate as a software in monitor device 148 or as a software in a remote server that serves the interface via HTML for example.

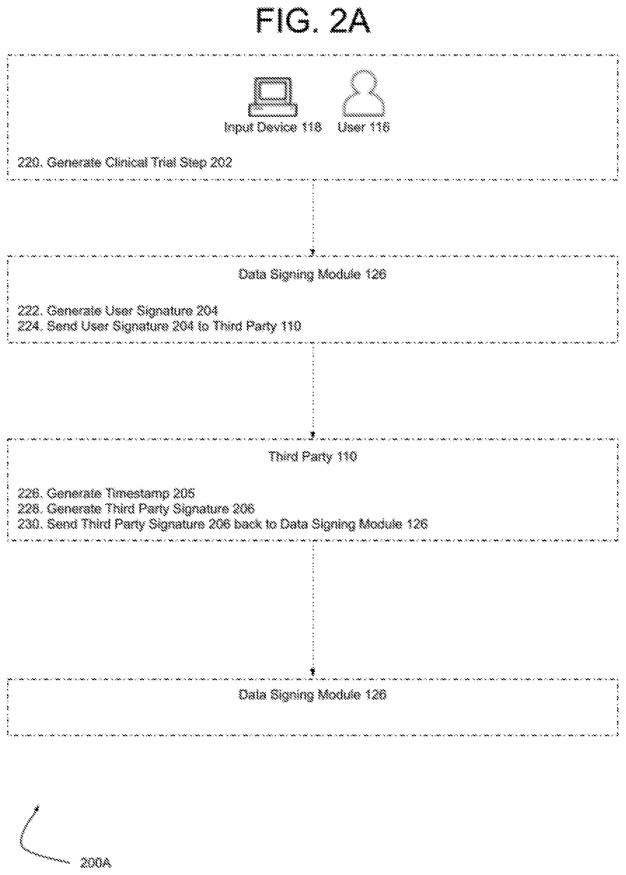

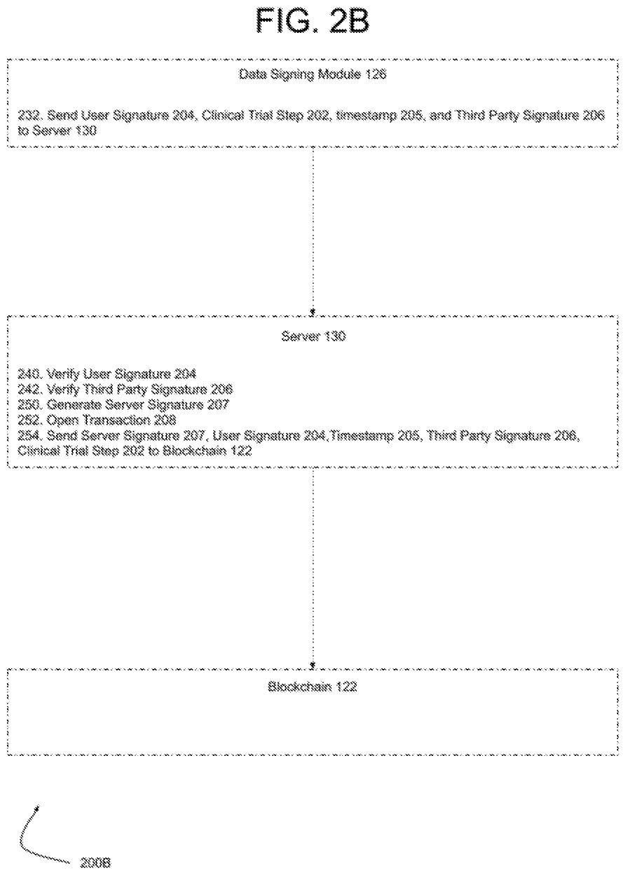

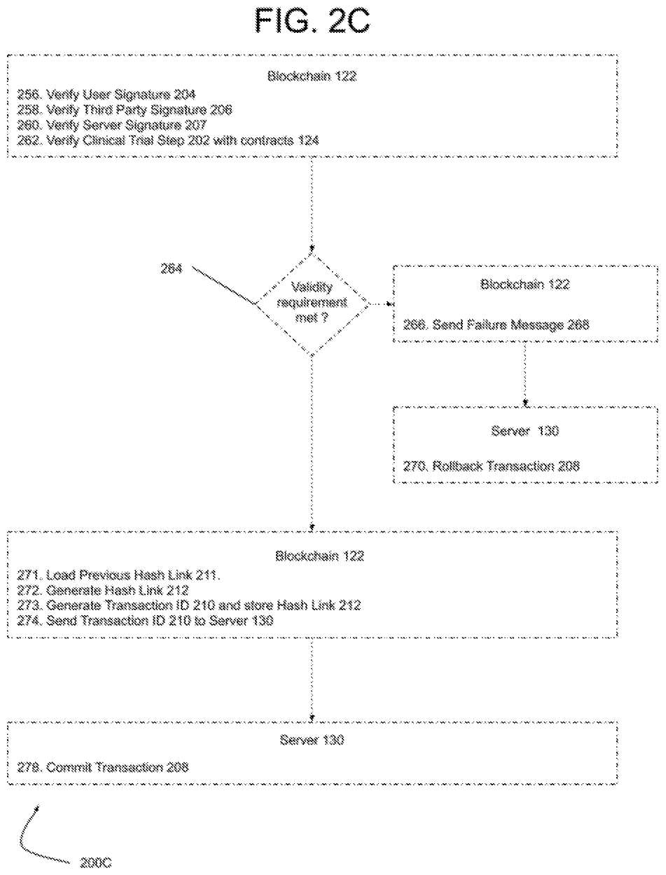

FIG. 2A, FIG. 2B and FIG. 2C are flowcharts illustrating an example of data flow between elements of FIG. 1.

FIG. 2A, FIG. 2B and FIG. 2C may contain similar numbered elements from FIG. 1.

Referring to FIG. 2A.

Flowchart 200A may include input device 118, user 116, data signing module 126, third party 110, clinical trial step 202, user signature 204. timestamp 205, third party signature 206, and steps 220, 222, 224, 226, 228 and 230.

Data signing module 126 may be used by user 116 with input device 118. Data signing module 126 may be used by user 116 with input device 118.

At step 220, user 116 may use input device 118 to form a clinical trial step 202.

Clinical trial step 202 may be as simple as a read request to initially form an interface graphic presented to user 116 or a more elaborate request like an action related to a clinical trial activity. These actions can vary for each user depending on their role, permissions, and phase of the clinical trial.

Some examples of actions include: A coordinator captures data related to a patient's vital status or reports an adverse event at a healthcare facility. A coordinator updates a status related to a query originated from a manager. A manager reports shipping of investigational medicine to a hospital.

At step 222, user 118 sends clinical trial step 202 to data signing module 126 to generate user signature 204.

Data signing module 126 can operate as a software in input device 118 or as a software in a remote server that serves the interface via. HTML for example. For example data signing module 126 can be located in smartphone, a remote server or personal computer. Clinical trial step 202 and user private key 128 may be provided to signing algorithm 114 to generate user signature 204. User signature 204 can be used to verify the data integrity of clinical step 202 (data integrity) and can be formally associated with user 116 (non-repudiation).

At step 224, data signing module 126 may send user signature 204 to third party 110 via communication network 150. Because third party 110 may only receive user signature 204, third party 110 may be a public blockchain, a private service, a software or other system.

At step 226, third party 110 generates timestamp 205 associated with current time.

At step 228, timestamp 205, third party private key 112 and user signature 204 are provided to signing algorithm 114 to generate third party signature 206. The resulting third party signature 206 may be used to verify the time at which user signature 204 was submitted to third party 110 (proof-of-time) while guaranteeing the privacy of clinical trial step 202. Therefore, data validity is improved by providing proof of time.

At step 230, third party signature 206 and timestamp 205 are sent back to data signing module 126 via public communication 150.

Referring to FIG. 2B,

Flowchart 200B may include data signing module 126, server 130, blockchain 122, user signature 204, third party signature 206, server signature 207, transaction 208, timetamp 205, and steps 232, 240, 242, 250, 252, and 254.

At step 232, user signature 204, clinical trial step 202, and third party signature 206 are sent to server 130 via communication network 150.

At step 240, user signature 204, clinical trial step 202 and user digital certificate 152 may be provided to signature verifying algorithm 144. Resulting output may accept or reject authenticity's claim of user signature 204 related to clinical trial step 202. Rejection of authenticity's claim of user signature 204 related to clinical trial step 202 may end the process.

At step 242, third party signature 206, timestamp 205, user signature 204, and third party digital certificate 154 may be provided to signature verifying algorithm 144. Rejection of authenticity's claim of third party signature 206 related to timestamp 205 and user signature 204 may end the process.

At step 250, server private key 132 and user signature 204 may be provided to signing algorithm 114 to generate server signature 207.

At step 252, transaction manager 134 may open a transaction 208 with database 136.

At step 254, server 130 sends clinical trial step 202, third party signature 206, timestamp 205, user signature 204 and server signature 207 to blockchain 122.

Referring to FIG. 2C,

Flowchart 200C may include blockchain 122, server 130, user signature 204, third party signature 206, server signature 207, clinical trial step 202, contracts 124, transaction 208, previous hash link 211, hash link 212, transaction ID 210, steps 256, 258, 260, 262, 266, 270, 271, 273, 274 and 278, and conditional block 264.

At step 256, user signature 204, clinical trial step 202 and user digital certificate 152 may be provided to signature verifying algorithm 144. Resulting output may accept or reject authenticity's claim of user signature 204 related to clinical trial step 202. User digital certificate 152 may be distributed by public key infrastructure 120 via communication network 150.

At step 258, third party signature 206, timestamp 205, user signature 204, and third party digital certificate 154 may be provided to signature verifying algorithm 144. Resulting output may accept or reject authenticity's claim of third party signature 206 related to timestamp 205 and user signature 204.

At step 260, server signature 207, third party signature 206, and server digital certificate 156 may be provided to signature verifying algorithm 144. Resulting output may accept or reject authenticity's claim of server signature 207 related to third party signature 206.

At step 262, clinical trial step 202 and timestamp 205 are provided to contracts 124 which may verify the validity of the request by checking the current phase of the trial and the rules defined by the protocol. As explained above, contracts 124 may be programmed by a programmer using the specification of blockchain 122.

Step 256, step 258, and step 260 may be delegated to contracts 124.

At conditional block 264, failures at step 256, 258, 260, or 262 may result in blockchain sending a failure message to server 130 at step 266.

At step 270, failure message 268 received by server 130 may result in a rolling back transaction 208. By rolling back transaction 208, clinical trial step 202 may result in datastore 136 rejecting an clinical activity.

At step 271, providing that contracts 124 validated clinical trial step 202 at step 262, blockchain loads previous hash link 211. Previous hash link 211 may correspond to the validity proof preceding clinical trial step 202. In this example for simplicity, we use one previous hash link 211. But it can be understood that previous hash link 211 is optional in the case if clinical trial step 202 doesn't depend on any previous step. Or it can be more than one in the case where clinical trial step 202 depends on multiple previous steps.

At step 272, third party signature 206 and previous hash link 211 are provided to hash algorithm 140 to generate hash link 212. As explained above, it can be understood that previous hash link 211 is optional in the case if clinical trial step 202 doesn't depend on any previous step. Or it can he more than one in the case where clinical trial step 202 depends on multiple previous steps.

At step 273, blockchain generates transaction ID 210 and stores hash link 212 in blockchain persistence layer. Transaction ID 210 represents the ID of the record used to store hash link 212, as well as a pointer to previous hash link 211. Transaction ID 210 may be used for later retrieval of data.

At step 274, blockchain 122 sends transaction ID 210 back to server 130.

At step 278, server 130 stores server signature 207, user signature 204, timestamp 205, third party signature 206, clinical trial step 202, and transaction ID 210 to datastore 136 and commits transaction 208.

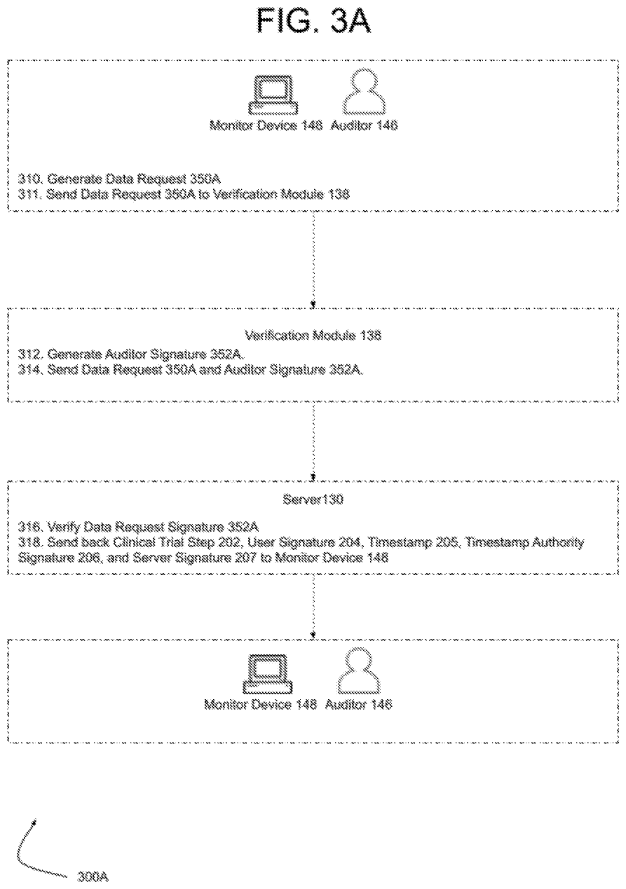

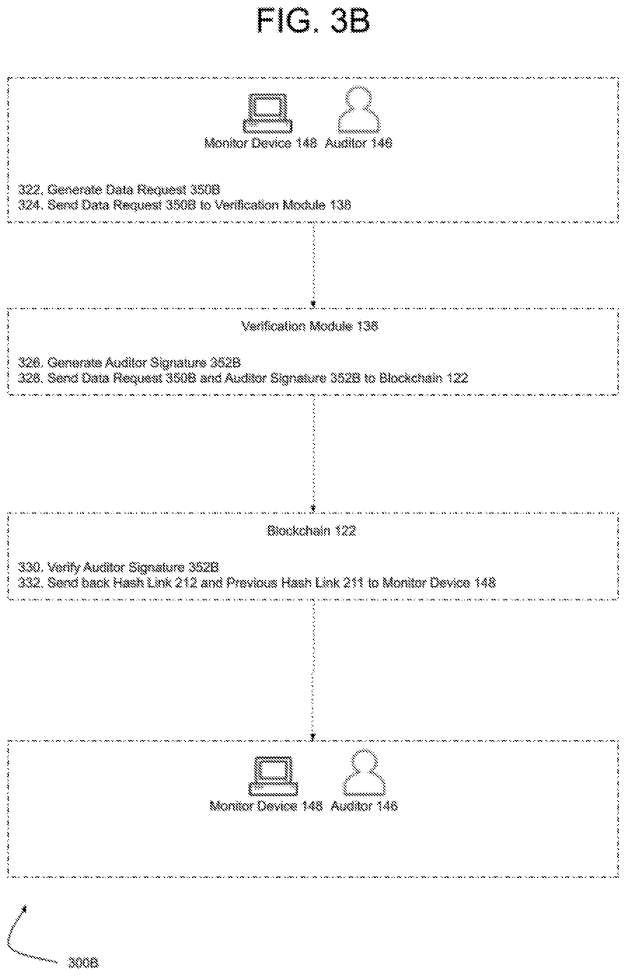

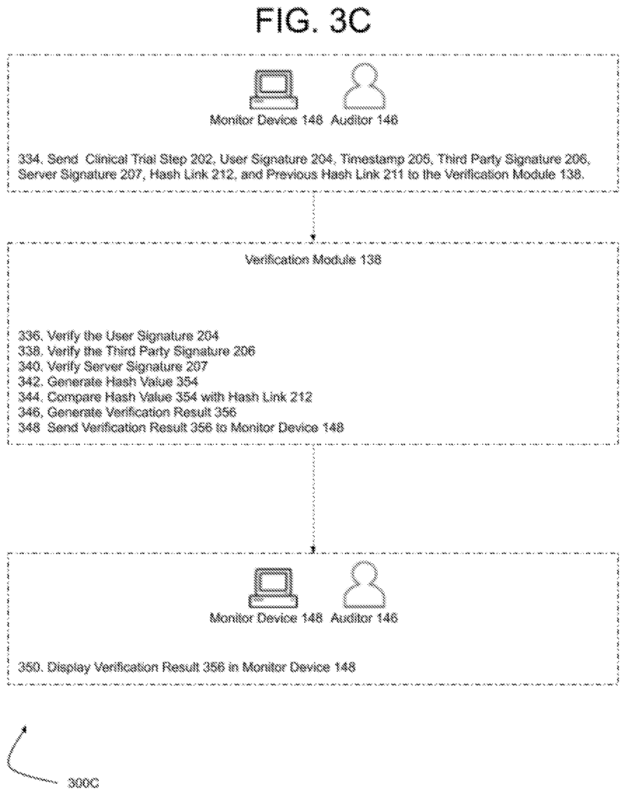

FIG. 3A, FIG. 39, and. FIG. 3C are flowcharts illustrating an example of data flow between elements of FIG. 1.

FIG. 3A and FIG. 3B may contain similar numbered elements from FIG. 1, FIG. 2A, FIG. 29 and FIG. 2C.

Referring to FIG. 3A.

Flowchart 300A may include monitor device 148, auditor 146, server 130, verification module 138, data request 350A, auditor signature 352A, clinical trial step 202, user signature 204, timestamp 205, third party signature 206, server signature 207, and steps 310, 311, 312, 314, 316, and 318.

Auditor 146 can use monitor device 148 and verification module 138 to verify the accuracy of data related to a clinical trial.

An auditor 146 is a person that might have the permissions to read and verify the data. Auditor 146 may be a representative of a regulatory authority. Auditor 146 may be a consultant that audits the quality of the clinical trial.

Monitor device 148 can be a personal computer, a smartphone or any other electronic device able to interact with the system

At step 310, auditor 146 uses monitor device 148 to generate data request 350A to server 130. Data request 350A may be a message sent over communication network 150 requesting data from server 130. Data requested may include clinical trial step 202, user signature 204, timestamp 205, third party signature 206, server 206, and transaction ID 210.

At step 311, data request 350A is sent to verification module 138.

At step 312, data request 350A and auditor private key 160 are provided to signing algorithm 114 to generate auditor signature 352A.

At step 314, data request 350A and auditor signature 352A is sent to server 130

At step 316, auditor signature 352A and timestamp auditor digital certificate 158 may be provided to signature verifying algorithm 144. Resulting output may accept or reject authenticity's claim of auditor signature 352A related to data request 350A. Rejection of authenticity's claim of auditor signature 352A related to data request 350A may end the process.

At step 318, server 130 may send back clinical trial step 202, user signature 204, timestamp 205, third party signature 206, server signature 207, and transaction ID 210 to monitor device 148.

Referring to FIG. 3B,

Flowchart 300B may include monitor device 148, auditor 146, verification module 138, blockchain 122, data request 3509, auditor signature 3529, hash link 212, previous hash link 211, and steps 322, 324, 326, 328, 330, and 332.

At step 322, auditor 146 uses monitor device 148 to generate data request 350B. Data request 350B can be a message sent over communication network 150 requesting data from blockchain 122. Data requested may include transaction ID 210, and may request hash link 212 and previous hash link 211. As explained above, it can be understood that previous hash link 211 is optional in the case if clinical trial step 202 doesn't depend on any previous step. Or it can be more than one in the case where clinical trial step 202 depends on multiple previous steps.

At step 324, auditor 146 uses monitor device 148 to send data request 350B to verification module 138.

At step 326, data request 3509 and auditor private key 160 are provided to signing algorithm 114 to generate auditor signature 352B.

At step 328, data request 350B and auditor signature 352B is sent to blockchain 122.

At step 330, data request 350B, auditor signature 352B, and timestamp auditor digital certificate 158 may be provided to signature verifying algorithm 144. Resulting output may accept or reject authenticity's claim of auditor signature 352B related to data request 350B. Rejection of authenticity's claim of auditor signature 3529 related to data request 350B may end the process.

At step 332, blockchain 122 sends back hash link 212 and previous hash link 211 to monitor device 148. As explained above, it can be understood that previous hash link 211 is optional in the case if clinical trial step 202 doesn't depend on any previous step. Or it can he more than one in the case where clinical trial step 202 depends on multiple previous steps.

Referring to FIG. 3C,

Flowchart 300C may include monitor device 148, auditor 146, verification module 138, clinical trial step 202, user signature 204, timestamp 205, third party signature 206, server signature 207, hash value 354, hash link 212, previous hash link 211,verification result 356, and steps 334, 336, 338, 340, 342, 344, 346, 348 and 350.

At step 334, auditor 146 sends clinical trial step 202, user signature 204, timestamp 205, third party signature 206, server signature 207, hash link 212, previous hash link 211 to verification module 138.

At step 336, user signature 204, clinical trial step 202, and user digital certificate 152 may be provided to signature verifying algorithm 144. Resulting output may accept or reject authenticity's claim of user signature 204 related to clinical trial step 202.

At step 338, third party signature 206, timestamp 205, user signature 204, and third party digital certificate 154 may be provided to signature verifying algorithm 144. Resulting output may accept or reject authenticity's claim of third party signature 206 related to timestamp 205 and user signature 204.

At step 340, server signature 207, third party signature 206, and server digital certificate 156 may be provided to signature verifying algorithm 144. Resulting output may accept or reject authenticity's claim of server signature 207 related to third party signature 206.

At step 342, third party signature 206 and previous hash link 211 are provided to hashing algorithm 114 to generate hash result 354. Hash result 354 may be a hash result of hashing algorithm 114 digitally representing third party signature 206 and previous hash link 211.As explained above, it can be understood that previous hash link 211 is optional in the case if clinical trial step 202 doesn't depend on any previous step. Or it can be more than one in the case where clinical trial step 202 depends on multiple previous steps.

At step 344, hash link 212 and hash result 354 are provided to comparison module 142. Comparison module 142 may provide a way to compare hash link 212 and hash result 354 . A positive result may indicate that hash link 212 and hash result 354 are identical which means that the data used to form hash link 212 and hash result 354 are identical. A negative result may indicate that hash link 212 and hash result 354 are different therefore data used to form hash result 354 and hash link 212 are different.

At step 346, result from step 336, 338, 340, and 344 are regrouped in a Verification result 356.

At step 348, verification result 356 is sent to monitor device 148.

St step 350, verification result 356 is displayed in monitor device 1.48.With verification result 356, auditor 146 may be able to assert data integrity, proof of actors, proof of anteriority, proof of context, or proof of process as explained above for clinical trial step 202. Auditor 146 may check validity of data related to a clinical trial by verifying the entire data store in datastore 136.

In one aspect, items such as blocks, modules, components, methods, operations, contracts, instructions , algorithms and functions have been described in terms of their functionality and may he implemented as hardware, software or a combination of software and hardware. Skilled artisans may implement the described functionality in varying ways.

While this specification contains many specifics, these should not be construed as limitations but rather as descriptions of particular implementations of the subject matter.

This specification should not be construed as limitations on the scope of what may be claimed but rather as descriptions of particular implementations of the subject matter.

### ASPECTS OF THE INVENTION

1. In one aspect of the invention, a system and methods are provided, comprising: a data signing module, a server, a third party, a blockchain, a verification module, a public key infrastructure, an input device, and a monitor device.
2. In a further aspect of the invention, the data signing module may be a server, a mobile phone, a smartphone, software, or any over electronic device configured to receive requests from a network.
3. In a further aspect of the invention, the verification result may be used in any electronic system.
4. In a further aspect of the invention, the private blockchain may be a permissioned blockchain.
5. In a further aspect of the invention, the third party signature and the timestamp may be sent directly to the server.
6. in a further aspect of the invention, the publicly accessible network may be a private network and may be restricted by one or multiple network rules.
7. In a further aspect of the invention, the data signing module may be colocated or part of the input device.
8. In a further aspect of the invention, the data signing module may be colocated or part of the server.
9. In a further aspect of the invention, the verification module may be collocated or part of the monitor device.
10. In a further aspect of the invention, the verification module may be collocated or part of the server.
11. In a further aspect of the invention, methods and systems may be used for any activity related to healthcare management.
12. In another aspect of the invention, the data signing device may authenticate an identity with the public key infrastructure or with a third party service.
13. In another aspect of the invention, the third party may be a server, a mobile phone, a smartphone, software, or any over electronic device configured to receive requests from a network.
14. In another aspect of the invention, the third party may be a blockchain-based distributed ledger
15. In another aspect of the invention, the third party may be a service.
16. In a further aspect of the invention, the server may a mobile phone, a smartphone, software, or any over electronic device configured to receive requests from a network.
17. In a further aspect of the invention, the data store may be a database with atomic transactions.
18. In a further aspect of the invention, the data store may be a database without transactions.
19. In a further aspect of the invention, communications may be served through an application program interface (API
20. In a further aspect of the invention, the blockchain may have multiple processing nodes.
21. In a further aspect of the invention, an electronic can be without any limitation a desktop computer, laptop computer, tablet, a mobile phone, a personal digital assistant, a mobile audio player, a global positioning system, a receiver, a video game console, a television, a or a software.

What is claimed is:

1. A method for securely managing clinical activity, the method comprising :
forming a value representing the clinical activity including a digital signature;
distributing the value to a blockchain having contracts;
and distributing the value to a third party;

2. The method of claim 1 wherein forming a value representing the clinical activity including a digital signature further comprising authenticating a digital signature associated with a digital certificate.

3. The method of claim 1, wherein distributing the value to a blockchain having contracts specifies a cryptocurrency fee.

4. The method of claim 1 further comprising storing the value in a datastore.

5. The method of claim 1 wherein distributing the value to a third party further notarizing the value with the third party.

6. The method of claim 1 wherein the third party is a blockchain based ledger.

7. The method of claim 1 wherein the value further represents a user digital signature associated with a user representing the clinical activity .

8. The method of claim 7 wherein the value further represents a third party digital signature associated with the third party representing a combination of the clinical activity, the user digital signature and a timestamp.

9. The method of claim 8 wherein the value further represents a code digital signature associated with the code used to form or collect the value representing a combination of the clinical activity, the user digital signature, a timestamp and the third party digital signature.

10. The method of claim 1 further comprising storing the value in the blockchain including providing the value to a computing node of plurality of computing nodes associated with the blockchain wherein the computer forms a block for addition to the blockchain, the block including a representation of the value.

11. The method of claim 1 wherein distributing the value to a blockchain having contracts further includes forming a value representing a proof associated with clinical activity including a combination of data integrity, actor non-repudiation, proof of anteriority, proof of context and proof of process.

12. The method of claim 11 wherein forming a value representing a proof further includes one or multiple proof associated with clinical activity.

13. The method of claim 11 further comprising storing the proof in the blockchain.

14. The method of claim 1 further comprising receiving a confirmation that the value has been validated by the contracts.

15. The method of claim 1 further comprising receiving a confirmation that the value has been stored in the datastore.

16. The method of claim 1 wherein forming a value representing the clinical activity includes forming a graph where a node represents a clinical step and an edge a dependency between two clinical steps.

17. The method of claim 1 wherein forming a value representing the clinical activity includes forming an audit trail.

18. A method for verifying a clinical trial comprising :
receiving a value representing clinical activity from a datastore including one or multiple digital signatures;
receiving a second value included in a transaction record associated with the value from a blockchain;
comparing the second value with the first value;
and verifying one or more digital signatures.

19. A system comprising :
means for forming a value representing the clinical activity including a digital signature;
means for authenticating a user;
means for distributing the value to a third party ;
means for distributing the value to a least a portion of a plurality of processing nodes of a blockchain having contracts;
and means for storing the value in a data store.

20. The system of claim 19 wherein contracts are configured to conform to one or more rules or regulations

21. The system of claim 19 wherein the means for transacting a value representing clinical activity is configured to perform a least one of: storing the value into the data store , or retrieving the value from the data store

22. Software for securely managing clinical activity, the software stored in a non-transitory form on a computer-readable medium and including instructions for causing a computing system to:
form a value representing the clinical activity including one or multiple digital signature;
distribute the value to a blockchain having contracts;
and distribute the value to a third party;
